(19) United States
(12) Patent Application Publication
(10) Pub. No.: US 2016/0340650 A1
(43) Pub. Date: Nov. 24, 2016

(54) BLOOD DERIVED IMMUNE STIMULATORY COMPOSITIONS

(71) Applicant: Batu Biologics, Inc., San Diego, CA (US)

(72) Inventors: Samuel C. Wagner, San Diego, CA (US); Thomas E. Ichim, San Diego, CA (US); Vladimir Bogin, San Diego, CA (US)

(73) Assignee: Batu Biologics, Inc., San Diego, CA (US)

(21) Appl. No.: 15/162,352

(22) Filed: May 23, 2016

Related U.S. Application Data
(60) Provisional application No. 62/164,750, filed on May 21, 2015.

(51) Int. Cl. C12N 5/0787

(52) U.S. Cl. CPC C12N 5/0642

(57) ABSTRACT

Neutrophil extracellular traps (NETS) are webs of DNA held together with immunogenic peptides, released by neutrophils subsequent to activation. NETS are the most potent stimulator of dendritic cells, monocytes and T cells given their ability to activate TLR3, TLR4, TLR7 and TLR9. The use of NETS for in vivo stimulation of immunity in a therapeutic sense has not been utilized due to fear of anti-DNA antibody formation and subsequent development of systemic lupus erythromatosis. The current invention provides means of safely generating NETS in vitro through peripheral blood utilizing clinically safe means such as yeast-derived component zymosan, isolating said NETS, utilizing said NETS to in vitro activate cytokine production from PBMC in vitro, and concentrating said cytokines. Cytokines generated by this methodology have superior ability to stimulate NK cells in vitro as compared to isolated NK stimulatory cytokines. The invention provides means of utilizing said “symphony of cytokines” to treat cancer and viral infections.

### CROSS-REFERENCE TO RELATED APPLICATION

This application claims the benefit of U.S. Provisional Application No. 62/164,750 filed on May 21, 2015, the contents of which are incorporated herein by reference in its entirety.

### BACKGROUND

Cancer has historically been treated with surgery, radiation, chemotherapy, and hormone therapy. More recently, advances in understanding of the immune system's role in cancer have led to immunotherapy becoming an important treatment approach. Cancer immunotherapy began with treatments that nonspecifically activated the immune system and had limited efficacy and/or significant toxicity. In contrast, new immunotherapy treatments can activate specific, important immune cells, leading to improved targeting of cancer cells, efficacy, and safety. Within the immunotherapy category, treatments have included cytokine therapies, antibody therapies, and adoptive cell therapies.

In 1986, interferon-alpha became the first cytokine approved for cancer patients. In 1992, interleukin-2, or IL-2, was the second approved cytokine in cancer treatment, showing efficacy in melanoma and renal cell cancer. IL-2 does not kill cancer cells directly, but instead nonspecifically activates and stimulates the growth of the body's own T cells which then combat the tumor. Although interferon-a, IL-2, and subsequent cytokine therapies represent important advances in cancer treatment, they are generally limited by toxicity and can only be used in a limited number of cancers and patients.

After cytokines set the stage for immunotherapy, antibody therapies represented the next significant advance, with targeted specificity and a generally better-tolerated side effect profile. Monoclonal antibodies, or mAbs, are designed to attach to proteins on cancer cells, and once attached, the mAbs can make cancer cells more visible to the immune system, block growth signals of cancer cells, stop new blood vessels from forming, or deliver radiation or chemotherapy to cancer cells. The first FDA approved mAb specifically for cancer was Rituxan in 1997, and since then, many other antibodies have received approval, including Herceptin, Avastin, Campath, Erbitux, and Vectibix. More recently, antibodies have been conjugated with cytotoxic drugs to increase activity. The first approved antibody drug conjugate was Mylotarg in 2000, followed by Adcetris in 2011 and Kadcycla in 2013.

The next important advance has been the development of antibodies that target T cell checkpoint pathways, which are means by which cancer cells are able to inhibit or turn down the body's immune response to cancer. These treatments have shown an ability to activate T cells, shrink tumors, and improve patient survival. In 2011, Yervoy became the first checkpoint inhibitor approved by the FDA. Recent clinical data from checkpoint inhibitors such as nivolumab and Keytruda have confirmed both the approach and the importance of T cells as promising tools for the treatment of cancer.

Despite these many advances, a significant unmet need in cancer still persists.

### SUMMARY

The invention provides means of therapeutically using neutrophil extracellular traps (NETS) for the purpose of therapeutic immune modulation. NETs are well known to be one of the most potent immune stimulators naturally produced by the body. In fact, the potency of NETS can be seen in that they are a major cause of autoimmunity. The autoimmune condition associated with NETS, systemic lupus erythromatosis is associated with large systemic production of interferon alpha by plasmacytoid dendritic cells. Interferon alpha is a potent activator of NK cells, which has been demonstrated to be effective against numerous types of cancers, as well as viral infections. Unfortunately, there are at least 21 known types of interferon alpha. This makes the genetic engineering production of interferon alpha extremely difficult. This is also associated with patient nonresponsiveness. The invention provides means of in vitro generating NETS using clinically relevant stimulators of NETS.

### BRIEF DESCRIPTION OF THE FIGURES

The accompanying drawings incorporated herein and forming a part of the specification illustrate the example embodiments.

FIG. 1 shows the increase in neutrophil extracellular traps observed in zymosan treated cells.

FIG. 2 shows the increase in neutrophil extracellular traps observed in ozone treated cells.

FIG. 3 illustrates the induction of IFN-Alpha by NETS treated PBMC.

### DETAILED DESCRIPTION OF THE EMBODIMENTS

The invention provides means of therapeutically using neutrophil extracellular traps (NETS) for the purpose of therapeutic immune modulation. NETs are well known to be one of the most potent immune stimulators naturally produced by the body. In fact, the potency of NETS can be seen in that they are a major cause of autoimmunity. The autoimmune condition associated with NETS, systemic lupus erythromatosis is associated with large systemic production of interferon alpha by plasmacytoid dendritic cells. Interferon alpha is a potent activator of NK cells, which has been demonstrated to be effective against numerous types of cancers, as well as viral infections. Unfortunately, there are at least 21 known types of interferon alpha. This makes the genetic engineering production of interferon alpha extremely difficult. This is also associated with patient nonresponsiveness. The invention provides means of in vitro generating NETS using clinically relevant stimulators of NETS. Currently the majority of NET stimulation studies focus on PMA, which is a toxic molecule. In one embodiment, the invention describes means of stimulating NETS production by means of zymosan treatment of blood compositions containing neutrophils. Zymosan is a relatively innocuous substance that is a component of bakers yeast wall. In one embodiment, the invention provides means of stimulating NETS production by means of treatment of blood or isolated neutrophils with ozone therapy.

The administration of NETS in vivo for production of interferon alpha and other immunomodulatory effects is difficult due to the possibility of development of antinuclear antibodies and subsequently lupus. Accordingly the invention teaches the in vitro utilization of NETS to stimulate interferon production from PBMC of patients. In one embodiment NETS are generated from allogeneic cells, but utilized to stimulate interferon alpha from autologous blood. This way the personalized interferon alpha is produced in the supernatant. In another embodiment, NETS are produced from allogeneic blood and administered to blood allogeneic to the patient. Subsequent to activation of PBMC by NETS, culture supernatant is harvested and utilized as a “natural interferon”. Advantages of this approach include the fact that other cytokines are produced by this non-specific stimulation of PBMC ex vivo. Additionally, the cost of production utilizing the methods thought in this invention is substantially lower than administration of genetically engineered interferon alpha. Most importantly the means of immunomodulation disclosed are all generated using naturally occurring ingredients and therefore have a substantial level of safety.

### EXAMPLES

### Example 1

### Stimulation of Neutrophil Extracellular Trap Formation by Treatment with Zymosan

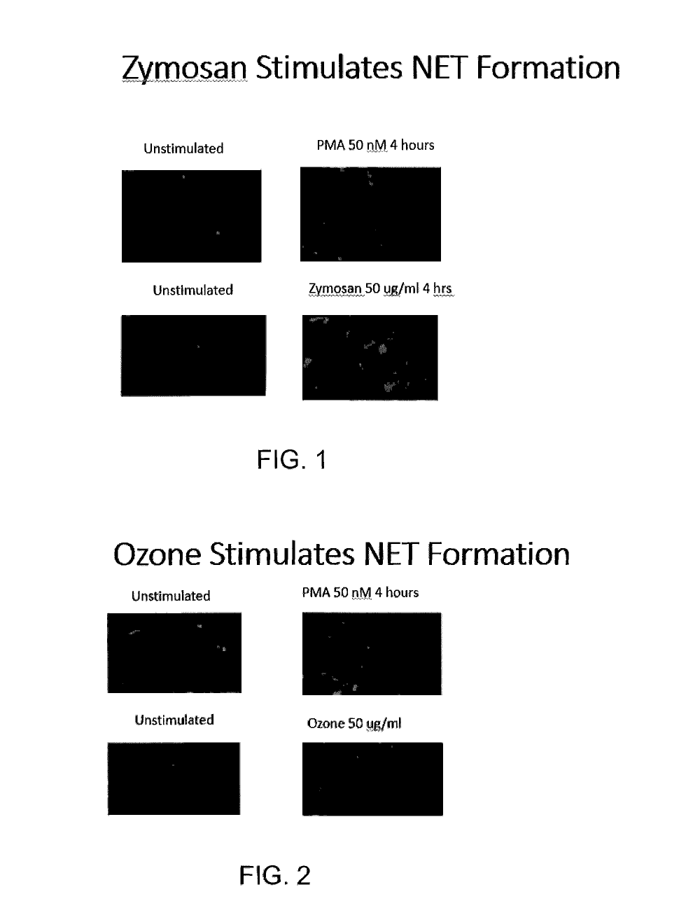

Blood was collected from health donors in EDTA tubes. Centrifugation of blood for 30 minutes at 1200 rpms was performed. Buffy coat was extracted by pipette and subsequently washed in phosphate buffered saline (PBS) 2 times. Buffy coat cells were incubated with 50 micrograms per ml of zymosan for 4 hours or as a positive control 50 nM of PMA for 4 hours. Cell incubation was performed in 3 ml volume in 6 well plates with a concentration of 1 million buffy coat cells per ml. Subsequent to incubation 5 ug/ml of Sytox Orange was added to the cultures and examination was performed under fluorescent microscopy. As shown in FIG. 1, increase in neutrophil extracellular traps was observed in zymosan treated cells.

### Example 2

### Stimulation of Neutrophil Extracellular Trap Formation by Treatment with Ozone

Blood was collected from health donors in EDTA tubes. Centrifugation of blood for 30 minutes at 1200 rpms was performed. Buffy coat was extracted by pipette and subsequently washed in phosphate buffered saline (PBS) 2 times. Buffy coat cells were incubated with 50 nM of PMA for 4 hours (positive control). Ozone treatment of blood was performed using a Vasogen Ozone generator at a concentration of 50 micrograms of ozone per ml of blood. Ozonation was performed by bubbling of ozone gas through the cellular mixture, said buffy coat cells in saline at a concentration of 1 million cells per ml. Four hours after ozonation 5 ug/ml of Sytox Orange was added to the cultures and examination was performed under fluorescent microscopy. As shown in FIG. 2, increase in neutrophil extracellular traps was observed in ozone treated cells.

### Example 3

### Stimulation of Interferon Alpha Production by Neutrophil Extracellular Traps

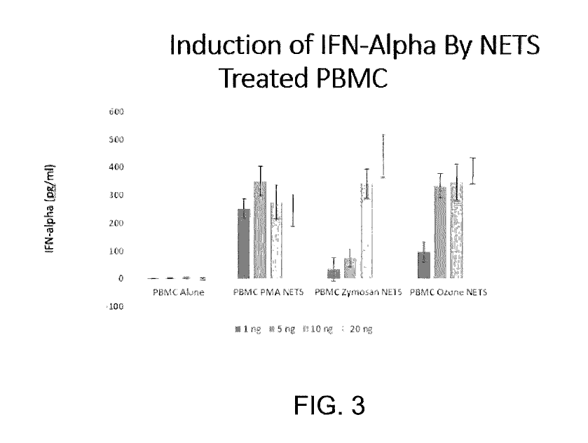

Neutrophil extracellular traps were isolated as described in Examples 1 and 2. Concentration of neutrophil extracellular traps was performed using the DNAFast kit from MP Research. A 48 hour culture of the indicated concentrations of neutrophil extracellular traps was performed with peripheral blood mononuclear cells (PBMC) at a concentration of 100,000 PBMC per well. Interferon alpha concentration was assessed by ELISA (R&D Systems) as shown in FIG. 3. Function of zymosan and ozone induced neutrophil extracellular traps was validated in that they both induced production of interferon alpha from PBMC.

1. A composition for the stimulation of immune responses, said composition derived by the steps of:
a) extracting a population of blood cells containing neutrophils;
b) contacting said population of blood cells containing neutrophils with an agent capable of stimulating neutrophil extracellular trap formation;
c) isolating said neutrophil extracellular traps;
d) contacting said neutrophil extracellular traps with one or more immune cells for a time period sufficient to induce activation of said immune cells;
e) collecting culture supernatant from said activated immune cells; and
f) concentrating said culture supernatant.

2. The composition of claim 1, wherein said population of blood containing neutrophils is buffy coat leukocytes.

3. The composition of claim 1, wherein said population of blood containing neutrophils is neutrophils isolated by a density gradient.

4. The composition of claim 3, wherein said density gradient is Dextran 500.

5. The composition of claim 3, wherein said density gradient is discontinuous Percoll gradient.

6. The composition of claim 1, wherein said agent capable of stimulating neutrophil extracellular trap formation is zymosan.

7. The composition of claim 6, wherein said zymosan is administered to said blood cells containing neutrophils at a concentration of 5-500 micrograms per ml of culture.

8. The composition of claim 6, wherein said zymosan is administered to said blood cells containing neutrophils at a concentration of 10-100 micrograms per ml of culture.

9. The composition of claim 6, wherein said zymosan is administered to said blood cells containing neutrophils at a concentration of 50 micrograms per ml of culture.

10. The composition of claim 1, wherein said agent capable of stimulating neutrophil extracellular trap formation is ozone.

11. The composition of claim 10, wherein said ozone is administered at a concentration that does not cause hemolysis.

12. The composition of claim 11, wherein said concentration of ozone is 0.1-100 micrograms per ml.

13. The composition of claim 12, wherein said concentration of ozone is 50 micrograms per ml.

14. The composition of claim 1, wherein said neutrophil extracellular traps are isolated by removal of cellular content and contacting remaining solution with a matrix capable of binding DNA.

15. The composition of claim 14, wherein said matrix capable of binding DNA is a silica-based matrix.

16. The composition of claim 15, wherein said silica based matrix is Maxabond.

17. The composition of claim 15, wherein said silica based matrix is 116540408 MP Binding Matrix.

18. The composition of claim 14, wherein DNA bound to said matrix is eluted by means of an elution media.

19. The composition of claim 18, wherein said elution media is a solution of 15% ethanol and 85% water.

20. The composition of claim 1, wherein said isolation of said neutrophil extracellular traps is performed by use of the FastDNA Spin kit.

21. The method of claim 1, wherein said isolated neutrophil extracellular trap is washed by ultracentrifugation in saline.

22. The method of claim 1, wherein said isolated neutrophil extracellular traps are contacted with peripheral blood mononuclear cells.

23. The method of claim 1, wherein said isolated neutrophil extracellular traps are contacted with a plasmacytoid DC cell line.

24. The method of claim 1, wherein said culture supernatant is collected after a culture ranging from 1-200 hours.

25. The method of claim 1, wherein said culture supernatant is collected after a culture ranging from 10-100 hours.

26. The method of claim 1, wherein said culture supernatant is collected after a culture of approximately 48 hours.

27. The method of claim 1, wherein said culture supernatant is concentrated by dialysis.

28. A method of stimulating neutrophil extracellular trap formation by contacting neutrophils with zymosan.

29. A method of stimulating neutrophil extracellular trap formation by contacting neutrophils with ozone.
